(19) United States
(12) Patent Application Publication
(10) Pub. No.: US 2025/0084061 A1
(43) Pub. Date: Mar. 13, 2025

(54) SULFONYL FUNCTIONAL COMPOUND, METHODS AND USES THEREOF

(71) Applicants: Hubei Biopesticide Engineering Research Centre, Wuhan (CN); Wuhan Pinestone Technology Co., Ltd., Wuhan (CN)

(72) Inventors: Shaoyong KE, Wuhan (CN); Fang LIU, Wuhan (CN); Longzhu BAO, Wuhan (CN); Zhigang ZHANG, Wuhan (CN); Lei YANG, Wuhan (CN); Chaoyang SONG, Wuhan (CN); Song YANG, Wuhan (CN); Kaimei WANG, Wuhan (CN); Wei FANG, Wuhan (CN); Hongqu WU, Wuhan (CN)

(73) Assignees: Hubei Biopesticide Engineering Research Centre, Wuhan (CN); Wuhan Pinestone Technology Co., Ltd., Wuhan (CN)

(21) Appl. No.: 18/648,410

(22) Filed: Apr. 28, 2024

(30) Foreign Application Priority Data
Sep. 7, 2023 (CN) 202311151393.1

(51) Int. Cl. C07D 401/12; A01N 43/40; A01N 43/52; A01N 43/707; A01P 1/00; A01P 3/00; A01P 7/04; C07D 213/36; C07D 213/61; C07D 213/82; C07D 235/30

(52) U.S. Cl. CPC C07D 401/12; A01N 43/40; A01N 43/52; A01N 43/707; A01P 1/00; A01P 3/00; A01P 7/04; C07D 213/36; C07D 213/61; C07D 213/82; C07D 235/30

(57) ABSTRACT

Provided is a compound selected from the group consisting of shown in formula (I), (II), (III), or (IV). Provided is a composition consisting of solvates or soluble salts formed by pharmaceutically acceptable solvents and the compound selected from the group shown in formula (I), (II), (III), or (IV). Provided is a pesticide consisting of a diluter and the compound selected from the group shown in formula (I), (II), (III), or (IV).

### CROSS-REFERENCE TO RELATED APPLICATIONS

This disclosure claims priority to Chinese Patent Application NO:202311151393.1, filed with China Intellectual Property Office on Sep. 7, 2023, the entire contents of which are incorporated herein by reference.

### TECHNICAL FIELD

This disclosure relates to agricultural chemicals. Specifically, this disclosure relates to sulfonyl functional compound, methods and uses thereof.

### BACKGROUND

The statements herein provide background information relevant to the present disclosure only and do not necessarily constitute prior art.

The agricultural chemicals are important agricultural production data, play an important role in the comprehensive treatment process of biological disasters such as agricultural diseases, insect pests and mice, and the like. The agricultural chemicals are widely applied to a plurality of fields such as plant protection, forestry, aquatic products, animal husbandry, sanitary pest prevention and control, and the like.

With the continuous change of many factors, such as the types of pests, the occurrence rules of pests, the structure of crop planting and the strategy of crop protection, the development and efficient utilization of new pesticides have become the key research direction of the application technology of agricultural chemicals.

### SUMMARY

In a first aspect, embodiments disclose a compound selected from the group consisting of:

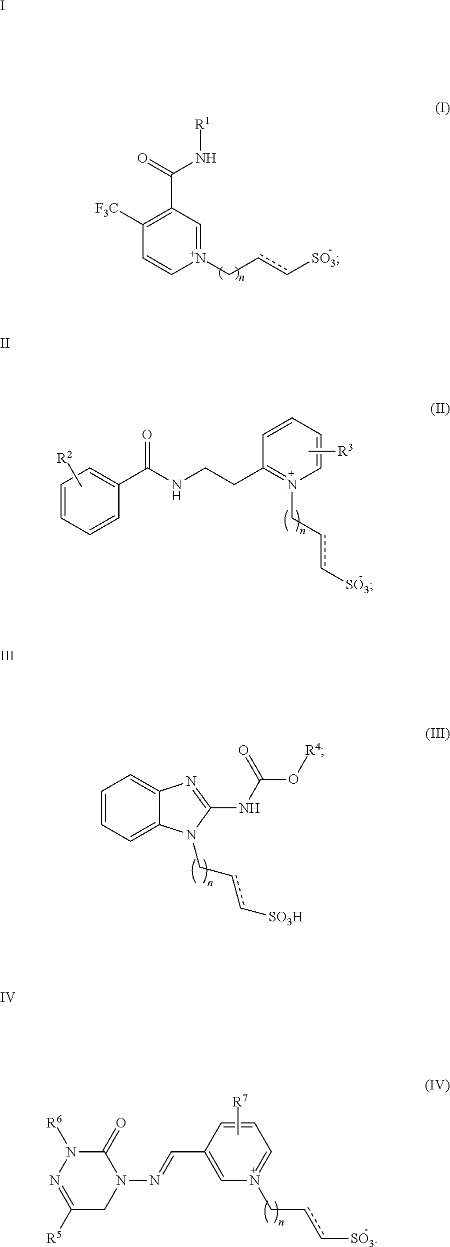

Therein, R1 is independently selected from the group consisting of CH₂CN, 4-CF₃OPhCH₂CH₂, 4-CF₃PhCH₂CH₂, 4-FPhCH₂CH₂, 4-CF₃OPhCH₂, CH₂CO₂Me, CH₂CONH₂, CH(CH₃)CO₂Me, CH(CH₃)CONH₂.

R2 is independently selected from the group consisting of H, 2-CF₃, 4-CF₃, 2-F, 2-CF₃O.

R3 is independently selected from the group consisting of H, F, Cl, Br, CF₃, CF₃O.

R4 is independently selected from the group consisting of CH₃, CH₂CH₃.

R5 is independently selected from the group consisting of CH₃, CH₂CH₃, Ph, 4-ClPh, 4-FPh, 4-CF₃Ph.

R6 is independently selected from the group consisting of H, COCH₃, COCH₂CH₃, COCF₃, CO₂CH₃, CO₂CH₂CH₃, CO₂tBu.

R7 is independently selected from the group consisting of H, F, Cl, Br, CF₃, CH₃, CF₃O, CN.

n is 1 or 2.

In a second aspect, embodiments disclose a composition consisting of solvates or soluble salts formed by the compound said in the first aspect and pharmaceutically acceptable solvents.

In third aspect, embodiments disclose a pesticide consisting of the compound said in the first aspect and a diluter.

In a fourth aspect, embodiments disclose a method of preparing the compounds said in the first aspect. The method includes alkylating a compound selected from the group consisting of (VI), (VII), (VIII), or (IX), with substituted sulfonolactone respectively.

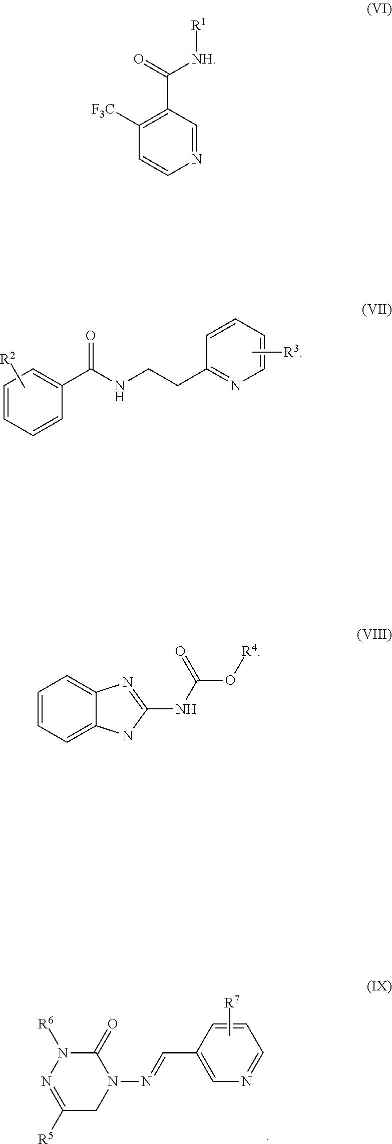

Therein, R1 is independently selected from the group consisting of CH₂CN, 4-CF₃OPhCH₂CH₂, 4-CF₃PhCH₂CH₂, 4-FPhCH₂CH₂, 4-CF₃OPhCH₂, CH₂CO₂Me, CH₂CONH₂, CH(CH₃)CO₂Me, CH(CH₃)CONH₂.

R2 is independently selected from the group consisting of H, 2-CF₃, 4-CF₃, 2-F, 2-CF₃O.

R3 is independently selected from the group consisting of H, F, Cl, Br, CF₃, CF₃O.

R4 is independently selected from the group consisting of CH₃, CH₂CH₃.

R5 is independently selected from the group consisting of CH₃, CH₂CH₃, Ph, 4-ClPh, 4-FPh, 4-CF₃Ph.

R6 is independently selected from the group consisting of H, COCH₃, COCH₂CH₃, COCF₃, CO₂CH₃, CO₂CH₂CH₃, CO₂tBu.

R7 is independently selected from the group consisting of H, F, Cl, Br, CF₃, CH₃, CF₃O, CN.

In a fifth aspect, embodiments disclose uses consisting of preparing drugs to kill biological pests, or drugs to control plant diseases with the compound said in the aspect above.

In a sixth aspect, embodiments disclose uses consisting of preparing drugs to kill biological pests, or drugs to control plant diseases with the composition said in the aspect above.

In a seventh aspect, embodiments disclose uses consisting of preparing drugs to kill biological pests, or drugs to control plant diseases with the pesticide said in the aspect above.

### BRIEF DESCRIPTION OF DRAWINGS

FIG. 1 shows the compound shown in formula (I-1) and it's preparing method according to Example 1.

FIG. 2 shows the compound shown in formula (I-2) and it's preparing method according to Example 2.

FIG. 3 shows the compound shown in formula (I-3) and it's preparing method according to Example 3.

FIG. 4 shows the compound shown in formula (I-4) and it's preparing method according to Example 4.

FIG. 5 shows the compound shown in formula (II-1) and it's preparing method according to Example 5.

FIG. 6 shows the compound shown in formula (II-2) and it's preparing method according to Example 6.

FIG. 7 shows the compound shown in formula (III-1) and it's preparing method according to Example 7.

FIG. 8 shows the compound shown in formula (III-2) and it's preparing method according to Example 8.

FIG. 9 shows the compound shown in formula (III-3) and it's preparing method according to Example 9.

FIG. 10 shows the compound shown in formula (IV-1) and it's preparing method according to Example 10.

FIG. 11 shows the compound shown in formula (IV-2) and it's preparing method according to Example 11.

FIG. 12 shows the compound shown in formula (IV-3) and it's preparing method according to Example 12.

FIG. 13 shows the compound shown in formula (IV-4) and it's preparing method according to Example 13.

FIG. 14 shows the compound shown in formula (IV-5) and it's preparing method according to Example 14.

FIG. 15 shows the compound shown in formula (IV-6) and it's preparing method according to Example 15.

FIG. 16 shows the compound shown in formula (IV-8) and it's preparing method according to Example 16.

### DETAILED DESCRIPTION OF THE EMBODIMENTS

The inventors of this disclosure creatively found that some compounds that are functional derivative containing sulfonic group, have good water solubility and obvious bactericidal or insecticidal activity. These compounds can be widely used in the integrated control of agricultural and forestry pests, and effectively improve the bioavailability of pests.

In some aspects, embodiments disclose a compound selected from the group consisting of (I)˜(IV).

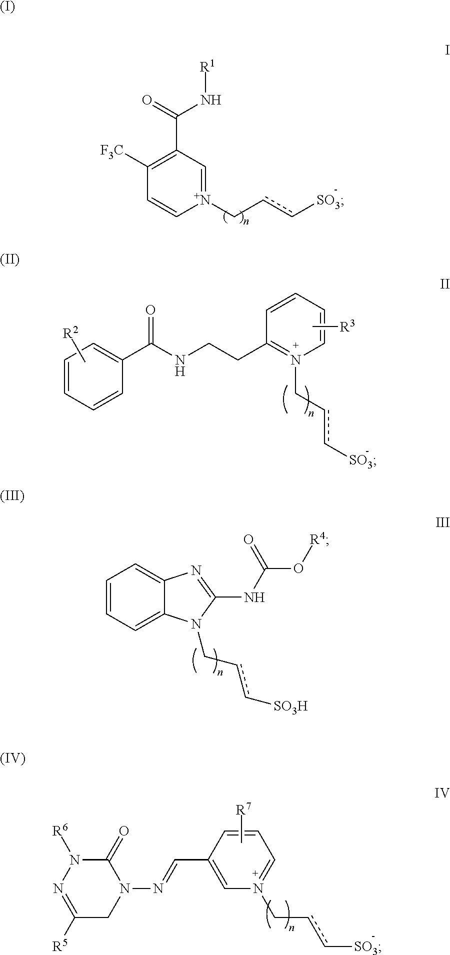

Therein, R1 is independently selected from the group consisting of CH₂CN, 4-CF₃OPhCH₂CH₂, 4-CF₃PhCH₂CH₂, 4-FPhCH₂CH₂, 4-CF₃OPhCH₂, CH₂CO₂Me, CH₂CONH₂, CH(CH₃)CO₂Me, CH(CH₃)CONH₂.

R2 is independently selected from the group consisting of H, 2-CF₃, 4-CF₃, 2-F, 2-CF₃O;

R3 is independently selected from the group consisting of H, F, Cl, Br, CF₃, CF₃O;

R4 is independently selected from the group consisting of CH₃, CH₂CH₃;

R5 is independently selected from the group consisting of CH₃, CH₂CH₃, Ph, 4-ClPh, 4-FPh, 4-CF₃Ph;

R6 is independently selected from the group consisting of H, COCH₃, COCH₂CH₃, COCF₃, CO₂CH₃, CO₂CH₂CH₃, CO₂tBu;

R7 is independently selected from the group consisting of H, F, Cl, Br, CF₃, CH₃, CF₃O, CN; [0065]n is 1 or 2.

As used herein, “tBu” refers to tertiary butyl. “Ph” refers to phenyl. “Me” refers to methyl. “Et” refers to ethyl. “Py” refers to pyridyl.

As used herein, “4-CF₃OPh” in “4-CF₃OPhCH₂CH₂” refers to that the fourth position of the benzene ring is “CF₃O”. “4-CF₃Ph” in “4-CF₃PhCH₂CH₂” refers to that the fourth position of the benzene ring is “CF₃”. “4-FPh” in “4-FPhCH₂CH₂” refers to that the fourth position of the benzene ring is “F”. “4-CF₃OPh” in “4-CF₃OPhCH₂” refers to that the fourth position of the benzene ring is “CF₃O”.

As used herein, “2-CF₃” refers to “CF₃” located at the second position of the benzene ring. “4-CF₃” refers to “CF₃” located at the fourth position of the benzene ring. “2-F” refers to “F” located at the second position of the benzene ring. “2-CF₃O” refers to “CF₃O” located at the second position of the benzene ring. “4-ClPh” refers to “Cl” located at the fourth position of the benzene ring. “4-FPh” refers to “F” located at the fourth position of the benzene ring. “4-CF₃Ph” refers to “CF₃” located at the fourth position of the benzene ring.

Nitrogen heterocyclic compounds are common molecular frameworks that are widely found in natural products and synthetic drugs. Due to their important physiological and biochemical functions, nitrogen heterocyclic compounds have very important applications in pharmaceutical chemistry, agricultural and food chemistry, biochemistry and polymer materials.

This disclosure provides functional derivatives obtained by sulfonic acid functionalization of nitrogen heterocyclic compounds, which have obvious bactericidal or insecticidal activity, and can be potentially applied to the integrated control of agricultural and forestry pests and diseases. The preparation process of these compounds is simple and easy, and they are functionally active substances with broad application prospects.

In some embodiments, the compound shown in formula (I) could be selected from the group consisting of:

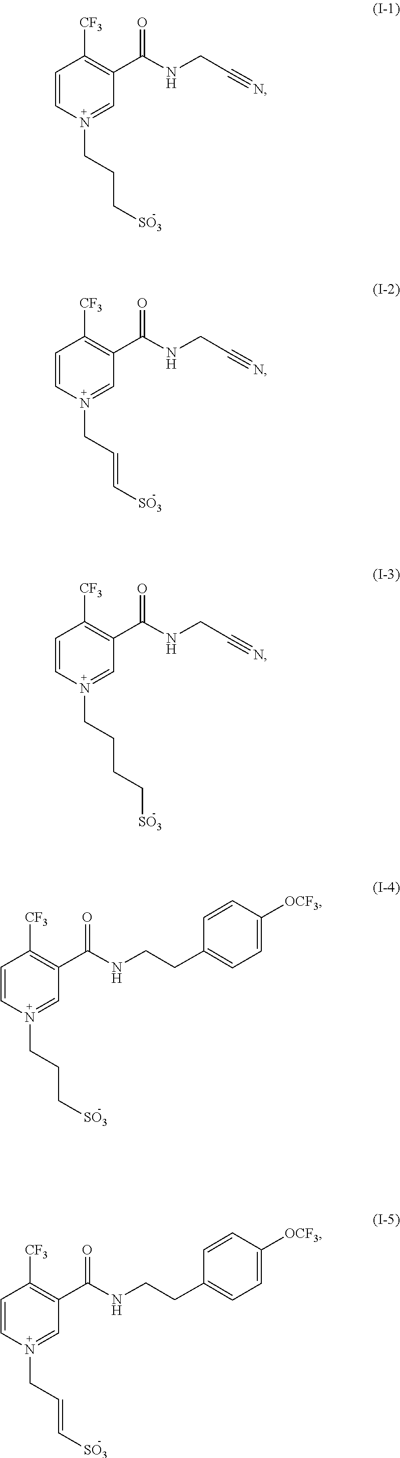

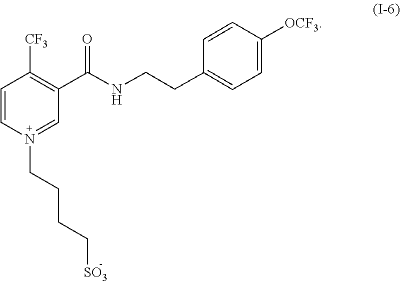

In some embodiments, the compound shown in formula (II) could be selected from the group consisting of:

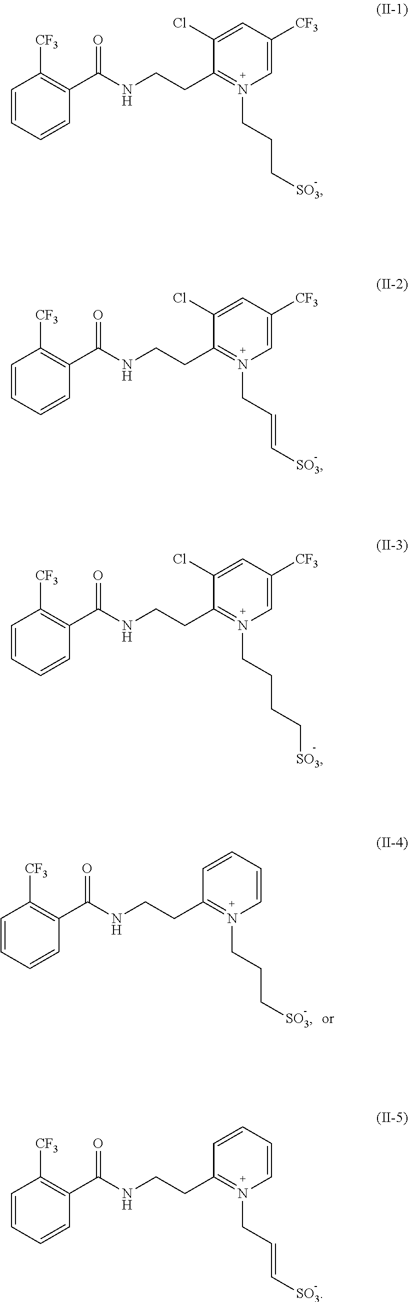

In some embodiments, the compound shown in formula (III) could be selected from the group consisting of:

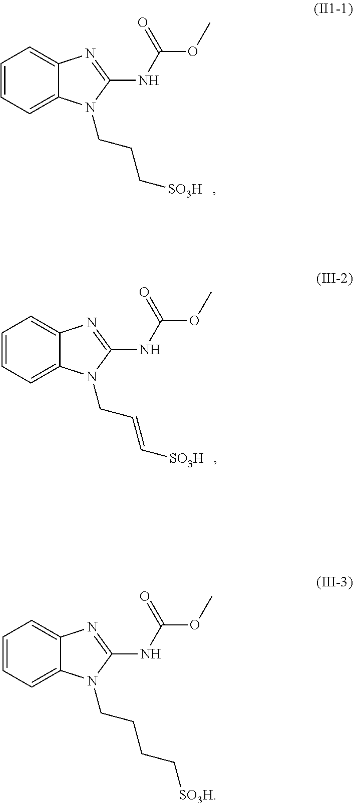 [SMILES: COC(=O)Nc1nc2ccccc2n1CCCS(=O)(=O)O ; COC(=O)Nc1nc2ccccc2n1C/C=C/S(=O)(=O)O ; COC(=O)Nc1nc2ccccc2n1CCCCS(=O)(=O)O]

In some embodiments, the compound shown in formula (IV) could be selected from the group consisting of:

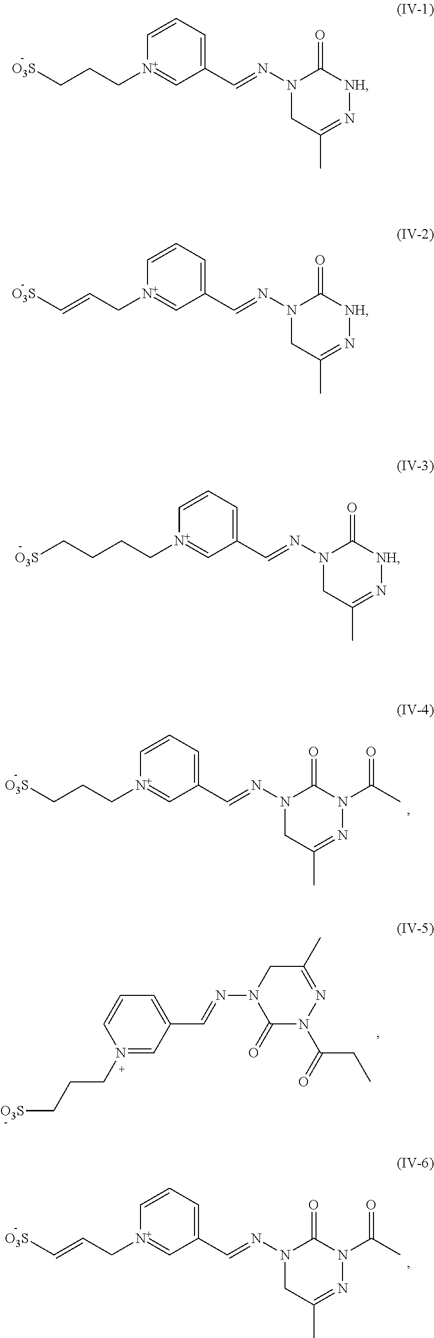

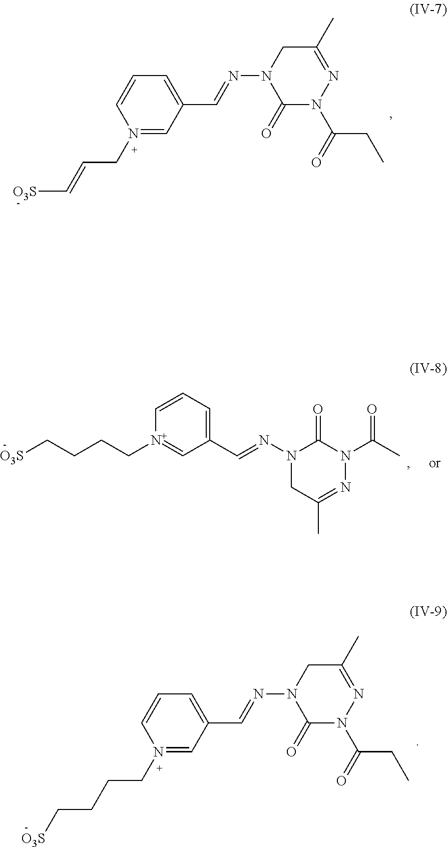

In another aspect, embodiments disclose a method of preparing the compound said in the first aspect. The method includes alkylating a compound selected from the group consisting of (VI), (VII), (VIII), or (IX), with substituted sulfonolactone respectively.

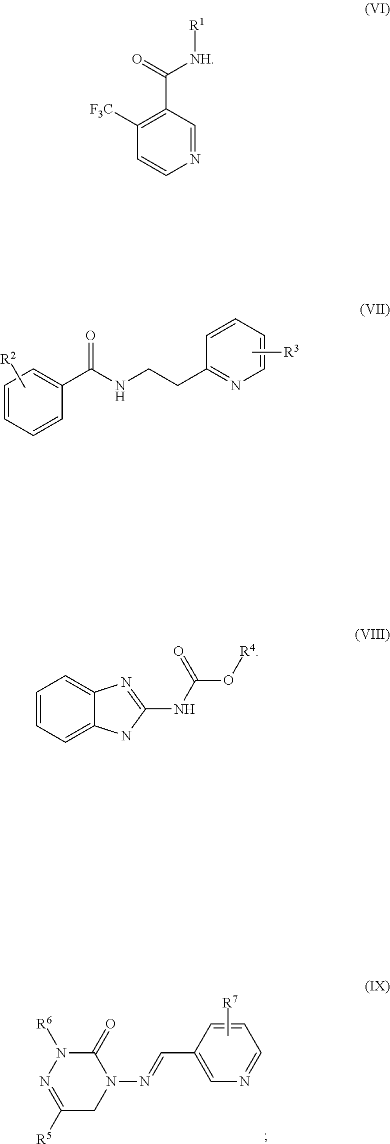

Therein, R1 is independently selected from the group consisting of CH₂CN, 4-CF₃OPhCH₂CH₂, 4-CF₃PhCH₂CH₂, 4-FPhCH₂CH₂, 4-CF₃OPhCH₂, CH₂CO₂Me, CH₂CONH₂, CH(CH₃)CO₂Me, CH(CH₃)CONH₂.

R2 is independently selected from the group consisting of H, 2-CF₃, 4-CF₃, 2-F, 2-CF₃O.

R3 is independently selected from the group consisting of H, F, Cl, Br, CF₃, CF₃O.

R4 is independently selected from the group consisting of CH₃, CH₂CH₃.

R5 is independently selected from the group consisting of CH₃, CH₂CH₃, Ph, 4-ClPh, 4-FPh, 4-CF₃Ph.

R6 is independently selected from the group consisting of H, COCH₃, COCH₂CH₃, COCF₃, CO₂CH₃, CO₂CH₂CH₃, CO₂tBu.

R7 is independently selected from the group consisting of H, F, Cl, Br, CF₃, CH₃, CF₃O, CN.

In some embodiments, the compound shown in formula (I) could be prepared by alkylating the compound shown in formula (VI) with various substituted sulfonolactone.

In some embodiments, the compound shown in formula (II) could be prepared by alkylating the compound shown in formula (VII) with various substituted sulfonolactone.

In some embodiments, the compound shown in formula (III) could be prepared by alkylating the compound shown in formula (VIII) with various substituted sulfonolactone.

In some embodiments, the compound shown in formula (IV) could be prepared by alkylating the compound shown in formula (IX) with various substituted sulfonolactone.

In some embodiments, the substituted sulfonolactone could select from the group consisting of 1,3-propane sultone, 1,4-butane sultone, 1,3-propenesultone, 3-hydroxypropanesultone, 3-chloropropanesultone, 3-fluoropropanesultone.

In some embodiments, the common reaction conditions of alkylation include suspending the substrate in the reaction solvent, adding the catalyst under stirring, slowly raising the temperature for the reaction, and monitoring the reaction process by using TLC and LC-MS analysis.

In the preparation of any of the compounds shown in formulas (I) to (IV) provided with some embodiments, the solvents used include at least one of tetrahydrofuran, toluene, 1,4-dioxane, xylene, 1,2-dichloroethane, o-dichlorobenzene, and heptane.

In the preparation of any of the compounds shown in formulas (I) to (IV) provided with some embodiments, the catalysts used include at least one of sodium hydride, potassium tert-butoxide, sodium methoxide, and N,N-diisopropylethylamine.

In the preparation of any of the compounds shown in formulas (I) to (IV) provided with some embodiments, the reaction temperature may be controlled at 40˜120° C.

In some embodiments, the reaction conditions for alkylation include: tetrahydrofuran used as solvent, sodium hydride used as catalyst, and the reaction temperature controlled at 40˜80° C.

In some embodiments, the reaction conditions for alkylation include: tetrahydrofuran used as solvent, potassium tert-butoxide used as catalyst, and the reaction temperature controlled at 40˜80° C.

In some embodiments, the reaction conditions for alkylation include: toluene used as solvent, sodium hydride used as catalyst, and the reaction temperature controlled at 40˜110° C.

In some embodiments, the reaction conditions for alkylation include: 1,4-dioxane used as solvent, sodium hydride used as catalyst, and the reaction temperature controlled at 40˜110° C.

In some embodiments, the reaction conditions for alkylation include: xylene used as solvent, sodium hydride used as catalyst, and the reaction temperature controlled at 40˜120° C.

In some embodiments, the reaction conditions for alkylation include: tetrahydrofuran used as solvent, sodium methoxide used as catalyst, and the reaction temperature controlled at 40˜80° C.

In some embodiments, the reaction conditions for alkylation include: toluene used as solvent, the reaction temperature controlled at 80˜110° C., and with no catalyst.

In some embodiments, the reaction conditions for alkylation include: toluene used as solvent, N,N-diisopropylethylamine used as catalyst, and the reaction temperature controlled at 80˜110° C.

In some embodiments, the reaction conditions for alkylation include: 1,4-dioxane used as solvent, the reaction temperature controlled at 80˜110° C., and with no catalyst.

In some embodiments, the reaction conditions for alkylation include: 1,4-dioxane used as solvent, N,N-diisopropylethylamine used as catalyst, and the reaction temperature controlled at 80˜110° C.

In some embodiments, the reaction conditions for alkylation include: 1,2-dichloroethane used as solvent, the reaction temperature controlled at 80˜90° C., and with no catalyst.

In some embodiments, the reaction conditions for alkylation include: xylene used as solvent, the reaction temperature controlled at 80˜120° C., and with no catalyst.

In some embodiments, the reaction conditions for alkylation include: xylene used as solvent, N,N-diisopropylethylamine used as catalyst, and the reaction temperature controlled at 80˜120° C.

In some embodiments, the reaction conditions for alkylation include: o-dichlorobenzene used as solvent, the reaction temperature controlled at 80˜120° C., and with no catalyst.

In some embodiments, the reaction conditions for alkylation include: o-dichlorobenzene used as solvent, N,N-diisopropylethylamine used as catalyst, and the reaction temperature controlled at 80˜120° C.

In some embodiments, the reaction conditions for alkylation include: heptane used as solvent, N,N-diisopropylethylamine used as catalyst, and the reaction temperature controlled at 80˜110° C.

As used herein, the compound shown in formula (VI), (VII), (VIII), or (IX) could be prepared with a variety of basic chemical raw materials by conventional organic synthesis methods.

In some aspect, embodiments disclose a composition consisting of solvates formed with the compound selected from the group shown in formula (I), (II), (III), or (IV), and pharmaceutically acceptable solvents.

In some aspect, embodiments disclose a composition consisting of soluble salts formed with the compound selected from the group shown in formula (I), (II), (III), or (IV), and pharmaceutically acceptable solvents.

In another aspect, embodiments disclose a pesticide consisting of the compound selected from the group shown in formula (I), (II), (III), or (IV), and a diluter.

In some embodiments, the diluter could be selected from the group consisting of kaolin, clay, bentonite, clunch, diatomite, montmorillonite, active floridin, dolomite, quartz, calcium carbonate, talc, or magnesite.

In some embodiments, the diluter could be selected from the group consisting of polyvinyl alcohol, carboxymethyl cellulose, gum arabic, trace nutrients, natural or synthetic polymers in powder, granule or emulsion form.

In some embodiments, the pesticide could be made into water emulsion, suspension agent, wettable powder, water dispersion granule and other different preparations.

In another aspect, embodiments disclose uses consisting of preparing drugs to kill biological pests, or drugs to control plant diseases with the compound said in the aspect above.

In another aspect, embodiments disclose uses consisting of preparing drugs to kill biological pests, or drugs to control plant diseases with the composition said in the aspect above.

In another aspect, embodiments disclose uses consisting of preparing drugs to kill biological pests, or drugs to control plant diseases with the pesticide said in the aspect above.

In some embodiments, the pests could be selected from the group consisting of Lipaphis erysimi, Aphis laburniKaltenbach, Rhopalosiphum maidis (Fitch), Aphis craccivora Koch., Aphis gossypii Glover, Schizaphis graminum, Sitobion avenae (Fabricius), Myzus persicae (Sulzer), Brevicoryne brassicae (Linnaeus), Bemisia tabaci Gennadius, Laodelphax striatellus (Fallen), Nilaparvata lugens (Stal), Trialeurodes vaporariorum, Sogatella furcifera (Horváth), Diaphorina citri Kuwayama., Psylla Chinesis YangetLi, Nephotettix bipunctatus (Fabricius), Cicadella viridis, Euttetix teneilus, Empoasca fabae, Stephanitis chinensis Drake, Helopeltis fasciaticollis Poppius, Plutella xylostella (Linnaeus), Spodoptera exigua Hübner, Spodoptera litura Fabricius, Helicoverpa armigera (Hübner), Helicoverpa assulta (Guenée, 1852), Meloidogyne, Hercinothrips femoralis, or Stenchaetothrips biformis (Bagnall).

In some embodiments, the plant diseases could be selected from the group consisting of cotton verticillium wilt, wheat scab, watermelon wilt, rice sheath wilt, wheat glume blight, gray mold, rape black shank, pear black spot, tomato clavicola leaf spot, bacterial blight, rice blast, gum spore anthracnose, melon and fruit rot, watermelon cataplecta, apple orbicula, bacterial wilt, soft rot, diseases caused by Fusarium oxysporum, Phytophthora capsicum, Fusarium graminis, Streptospora alternaria, or Enterobacteriaceae.

The sulfonyl functional compound, methods and uses are further described in the following examples. Unless otherwise specified, the technical means employed in these examples are conventional means well known to the skilled in the art. Unless otherwise specified, the reagents, methods and equipment used in the invention are conventional.

### Example 1

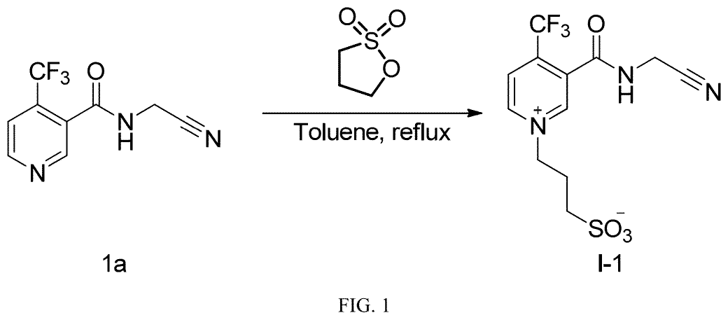

As shown in FIG. 1, example 1 provided a sulfonyl functional compound (I-1) and it's preparing method.

The method included: dissolving 1 mmol of substrate, N-(cyanomethyl)-5-(trifluoromethyl) nicotinamide (1a) in 10 mL of toluene; adding 1.1 mmol of 1,3-propane sultone into the solute under stirring; heating to 110-120° C. for thermal insulation reaction; monitoring with TLC until complete transformation of the substrate; and then cooling the reaction system to room temperature; evaporating a large amount of solvent from the reaction system under reduced pressure; precipitating solid from the cooling system, filtering under diminished pressure, washing the solid with a small amount of cold methanol, and vacuum drying to obtain the target compound I-1. The yield of compound I-1 is 74%. MS(ESI) m/z 352.27 (M+H)+, calcd. for C₁₂H₁₂F₃N₃O₄S m/z=351.05.

### Example 2

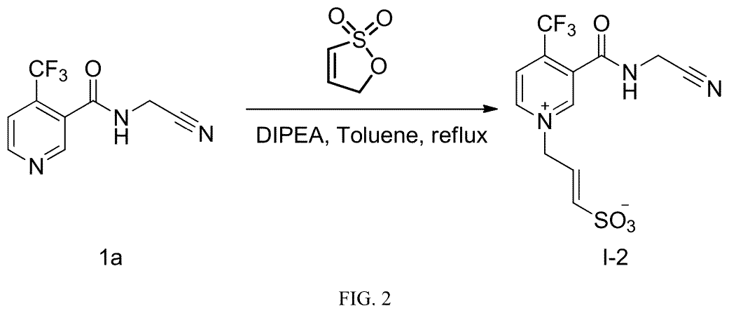

As shown in FIG. 2, example 2 provided a sulfonyl functional compound (I-2) and it's preparing method.

The method included: dissolving 1 mmol of substrate, N-(cyanomethyl)-5-(trifluoromethyl) nicotinamide (1a) in 10 mL of toluene; adding 1.1 mmol of prop-1-ene-1,3-sultone into the solute under stirring; adding 0.1 mmol of N,N-diisopropylethylamine (DIPEA) followed; slowly heating to 100˜110° C. for thermal insulation reaction; monitoring with TLC until complete transformation of the substrate; and then cooling the reaction system to room temperature; evaporating a large amount of solvent from the reaction system under reduced pressure; precipitating solid from the cooling system, filtering under diminished pressure, washing the solid with a small amount of cold methanol, and vacuum drying to obtain the target compound I-2. The yield of compound I-2 is 68%. MS(ESI) m/z 350.20 (M+H)+, calcd. for C₁₂H₁₀F₃N₃O₄S m/z=349.03.

### Example 3

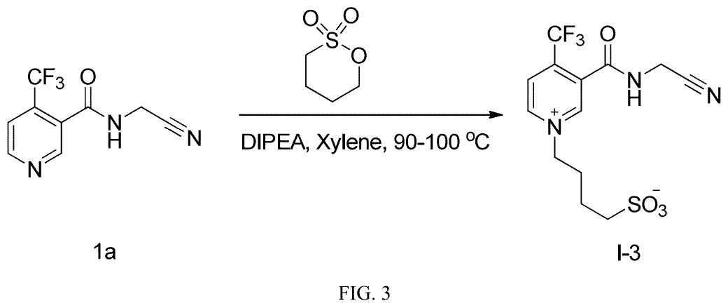

As shown in FIG. 3, example 3 provided a sulfonyl functional compound (I-3) and it's preparing method.

The method included: dissolving 1 mmol of substrate, N-(cyanomethyl)-5-(trifluoromethyl) nicotinamide (1a) in 10 mL of toluene; adding 1.1 mmol of 1,4-butane sultone into the solute under stirring; adding 0.1 mmol of N,N-diisopropylethylamine (DIPEA) followed; slowly heating to 90˜110° C. for thermal insulation reaction; monitoring with TLC until complete transformation of the substrate; and then cooling the reaction system to room temperature; evaporating 2/3 of the solvent from the reaction system under reduced pressure; precipitating solid from the cooling system, filtering under diminished pressure, washing the solid with a small amount of cold ethanol, and vacuum drying to obtain the target compound I-3. The yield of compound I-3 is 70%. MS(ESI) m/z 366.22 (M+H)+, calcd. for C₁₃H₁₄F₃N₃O₄S m/z=365.07.

### Example 4

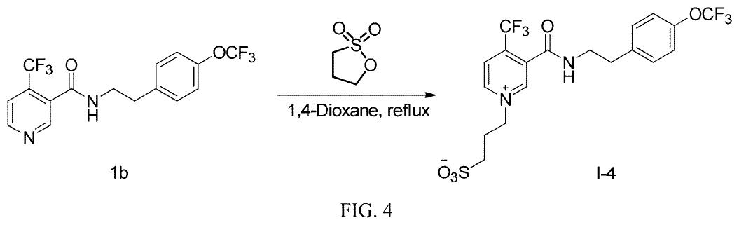

As shown in FIG. 4, example 4 provided a sulfonyl functional compound (I-4) and it's preparing method.

The method included: dissolving 1 mmol of substrate, N-(4-(trifluoromethoxy)phenethyl)-4-(trifluoromethyl)nicotinamide (1b) in 15 mL of 1,4-dioxane; adding 1.1 mmol of 1,3-propane sultone into the solute under stirring; slowly heating to 100˜110° C. for thermal insulation reaction; monitoring with TLC until complete transformation of the substrate; and then cooling the reaction system to room temperature; evaporating 1/2 of the solvent from the reaction system under reduced pressure; precipitating solid from the cooling system, filtering under diminished pressure, washing the solid with a small amount of cold isopropanol, and vacuum drying to obtain the target compound I-4. The yield of compound I-4 is 78%. MS(ESI) m/z 501.45 (M+H)+, calcd. for C₁₉H₁₈F₆N₂O₅S m/z=500.08.

### Example 5

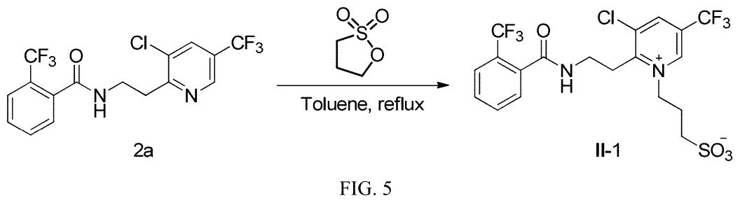

As shown in FIG. 5, example 5 provided a sulfonyl functional compound (II-1) and it's preparing method.

The method included: dissolving 1 mmol of substrate, N-(2-(3-chloro-5-(trifluoromethyl) pyridine-2-yl)ethyl)-2-(trifluoromethyl)benzamide (2a) in 20 mL of toluene; adding 1.1 mmol of 1,3-propane sultone into the solute under stirring; slowly heating to 110° C. for thermal insulation reaction; monitoring with TLC until complete transformation of the substrate; and then cooling the reaction system to room temperature; evaporating large amount of solvent from the reaction system under reduced pressure; precipitating solid from the cooling system, filtering under diminished pressure, washing the solid with a small amount of cold acetonitrile, and vacuum drying to obtain the target compound II-1. The yield of compound II-1 is 56%. MS(ESI) m/z 519.46 (M+H)+, calcd. for C₁₉H₁₇ClF₆N₂O₄S m/z=518.05.

### Example 6

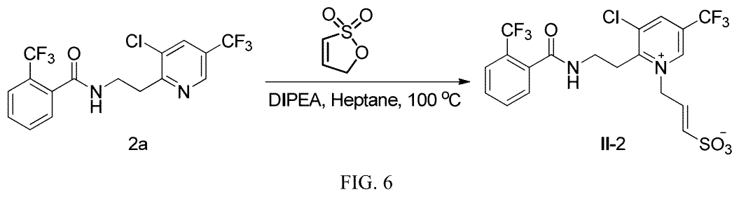

As shown in FIG. 6, example 6 provided a sulfonyl functional compound (II-2) and it's preparing method.

The method included: dissolving 1 mmol of substrate, N-(2-(3-chloro-5-(trifluoromethyl)pyridin-2-yl)ethyl)-2-(trifluoromethyl)benzamide (2a) in 20 mL of heptane; adding 1.1 mmol of prop-1-ene-1,3-sultone into the solute under stirring; adding 0.1 mmol of N,N-diisopropylethylamine (DIPEA) followed; slowly heating to 100° C. for thermal insulation reaction; monitoring with TLC until complete transformation of the substrate; and then cooling the reaction system to room temperature; evaporating 2/3 of solvent from the reaction system under reduced pressure; precipitating solid from the cooling system, filtering under diminished pressure, washing the solid with a small amount of cold ethanol, and vacuum drying to obtain the target compound II-1. The yield of compound II-2 is 63%. MS(ESI) m/z 517.32 (M+H)+, calcd. for C₁₉H₁₅ClF₆N₂O₄S m/z=516.03.

### Example 7

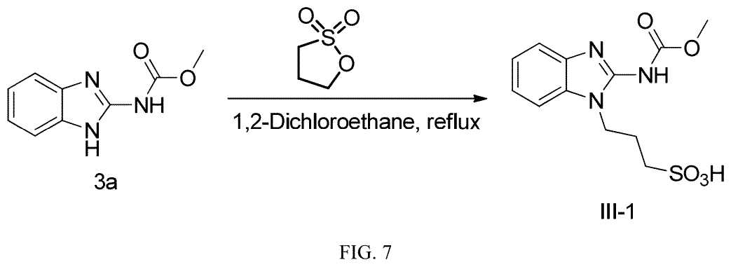

As shown in FIG. 7, example 7 provided a sulfonyl functional compound (III-1) and it's preparing method.

The method included: dissolving 1 mmol of substrate, N-(2-benzimidazolyl)methyl carbamate (3a) in 15 mL of dichloroethane; adding 1.1 mmol of 1,3-propane sultone into the solute under stirring; slowly heating to 80˜90° C. for thermal insulation reaction; monitoring with TLC until complete transformation of the substrate; and then cooling the reaction system to room temperature; evaporating 1/2 of solvent from the reaction system under reduced pressure; precipitating solid from the cooling system, filtering under diminished pressure, washing the solid with a small amount of cold ethanol, and vacuum drying to obtain the target compound III-1. The yield of compound III-1 is 85%. MS(ESI) m/z 314.09 (M+H)+, calcd. for C₁₂H₁₅N₃O₅S m/z=313.07.

### Example 8

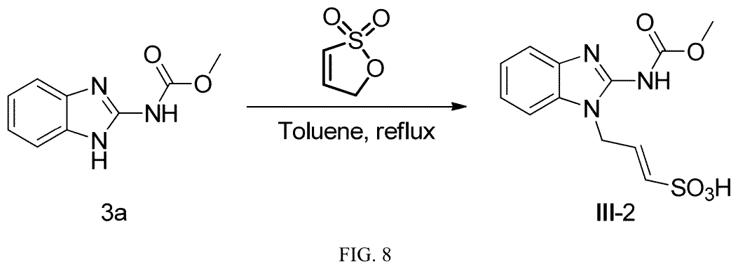

As shown in FIG. 8, example 8 provided a sulfonyl functional compound (III-2) and it's preparing method.

The method included: dissolving 1 mmol of substrate, N-(2-benzimidazolyl)methyl carbamate (3a) in 20 mL of toluene; adding 1.1 mmol of prop-1-ene-1,3-sultone into the solute under stirring; slowly heating to 110° C. for thermal insulation reaction; monitoring with TLC until complete transformation of the substrate; and then cooling the reaction system to room temperature; evaporating large amount of toluene from the reaction system under reduced pressure; precipitating solid from the cooling system, filtering under diminished pressure, washing the solid with a small amount of cold acetonitrile, and vacuum drying to obtain the target compound III-2. The yield of compound III-2 is 88%. MS(ESI) m/z 312.20 (M+H)+, calcd. for C₁₂H₁₃N₃O₅S m/z=311.06.

### Example 9

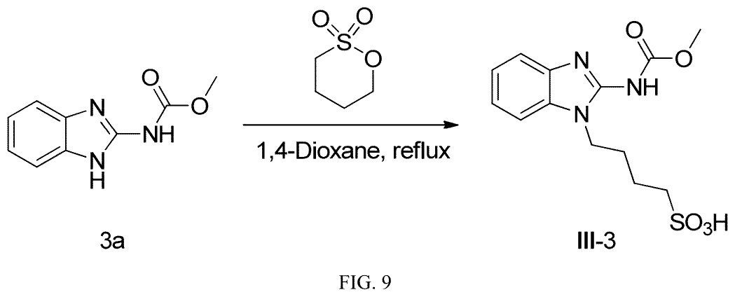

As shown in FIG. 9, example 9 provided a sulfonyl functional compound (III-3) and it's preparing method.

The method included: dissolving 1 mmol of substrate, N-(2-benzimidazolyl)methyl carbamate (3a) in 20 mL of 1,4-dioxane; adding 1.1 mmol of 1,4-butane sultone into the solute under stirring; slowly heating to 100-105° C. for thermal insulation reaction; monitoring with TLC until complete transformation of the substrate; and then cooling the reaction system to room temperature; evaporating 2/3 of solvent from the reaction system under reduced pressure; precipitating solid from the cooling system, filtering under diminished pressure, washing the solid with a small amount of cold acetonitrile, and vacuum drying to obtain the target compound III-3. The yield of compound III-3 is 91%. MS(ESI) m/z 328.23 (M+H)+, calcd. for C₁₃H₁₇N₃O₅S m/z=327.09.

### Example 10

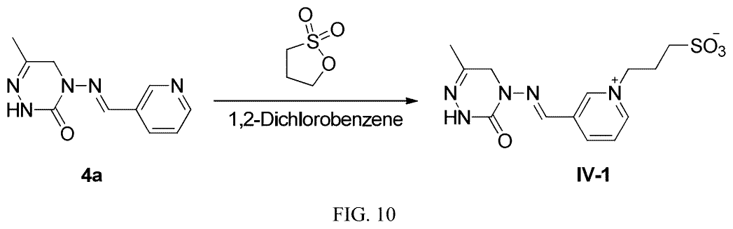

As shown in FIG. 10, example 10 provided a sulfonyl functional compound (IV-1) and it's preparing method.

The method included: dissolving 1 mmol of substrate, 6-methyl-4-((pyridin-3-ylmethylene)amino)-4,5-dihydro-1,2,4-triazin-3(2H)-one (4a) in 20 mL of o-dichlorobenzene; adding 1.1 mmol of 1,3-propane sultone into the solute under stirring; slowly heating to 110-120° C. for thermal insulation reaction; monitoring with TLC until complete transformation of the substrate; and then cooling the reaction system to room temperature; filtering under diminished pressure, washing the solid from the filter residue with a small amount of cold ethanol, and vacuum drying to obtain the target compound IV-1. The yield of compound IV-1 is 83%. MS(ESI) m/z 340.38 (M+H)+, calcd. for C₁₃H₁₇N₅O₄S m/z=339.10.

### Example 11

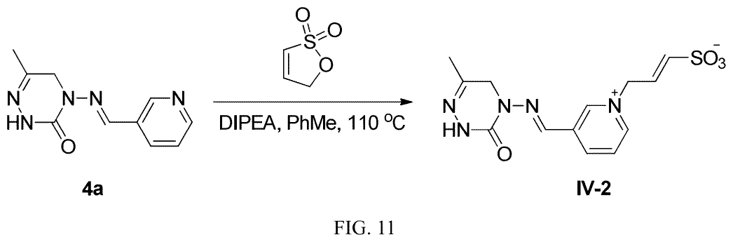

As shown in FIG. 11, example 11 provided a sulfonyl functional compound (IV-2) and it's preparing method.

The method included: dissolving 1 mmol of substrate, 6-methyl-4-((pyridin-3-ylmethylene)amino)-4,5-dihydro-1,2,4-triazin-3(2H)-one (4a) in 20 mL of toluene; adding 1.1 mmol of prop-1-ene-1,3-sultone into the solute under stirring; adding 0.1 mmol of N,N-diisopropylethylamine (DIPEA) followed; slowly heating to 110° C. for thermal insulation reaction; monitoring with TLC until complete transformation of the substrate; and then cooling the reaction system to room temperature; evaporating 2/3 of solvent from the reaction system under reduced pressure; precipitating solid from the cooling system, filtering under diminished pressure, washing the solid with a small amount of cold ethanol, and vacuum drying to obtain the target compound IV-2. The yield of compound IV-2 is 86%. MS(ESI) m/z 338.22 (M+H)+, calcd. for C₁₃H₁₅N₅O₄S m/z=337.08.

### Example 12

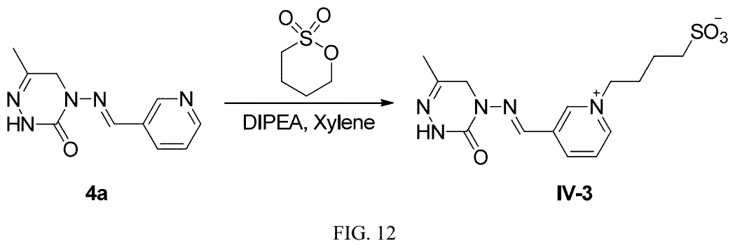

As shown in FIG. 12, example 12 provided a sulfonyl functional compound (IV-3) and it's preparing method.

The method included: dissolving 1 mmol of substrate, 6-methyl-4-((pyridin-3-ylmethylene)amino)-4,5-dihydro-1,2,4-triazin-3(2H)-one (4a) in 15 mL of xylene; adding 1.1 mmol of 1,4-butane sultone into the solute under stirring; adding 0.1 mmol of N,N-diisopropylethylamine (DIPEA) followed; slowly heating to 110-120° C. for thermal insulation reaction; monitoring with TLC until complete transformation of the substrate; and then cooling the reaction system to room temperature; filtering under diminished pressure; washing the solid from the filter residue with a small amount of cold ethanol, and vacuum drying to obtain the target compound IV-3. The yield of compound IV-3 is 85%. MS(ESI) m/z 354.25 (M+H)+, calcd. for C₁₄H₁₉N₅O₄S m/z=353.12.

### Example 13

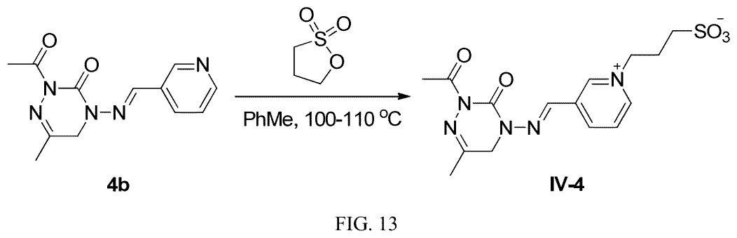

As shown in FIG. 13, example 13 provided a sulfonyl functional compound (IV-4) and it's preparing method.

The method included: dissolving 1 mmol of substrate, 2-acetyl-6-methyl-4-((pyridin-3-ylmethylene)amino)-4,5-dihydro-1,2,4-triazin-3(2H)-one (4b) in 20 mL of toluene; adding 1.1 mmol of 1,3-propane sultone into the solute under stirring; slowly heating to 100˜110° C. for thermal insulation reaction; monitoring with TLC until complete transformation of the substrate; and then cooling the reaction system to room temperature; filtering under diminished pressure; washing the solid from the filter residue with a small amount of cold ethanol, and vacuum drying to obtain the target compound IV-4. The yield of compound IV-4 is 88%. MS(ESI) m/z 382.43 (M+H)+, calcd. for C₁₅H₁₉N₅O₅S m/z=381.11.

### Example 14

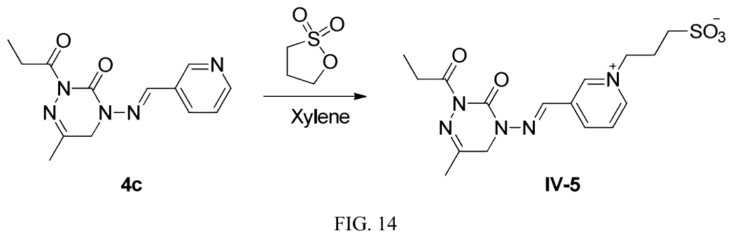

As shown in FIG. 14, example 14 provided a sulfonyl functional compound (IV-5) and it's preparing method.

The method included: dissolving 1 mmol of substrate, 6-methyl-2-propionyl-4-((pyridin-3-ylmethylene)amino)-4,5-dihydro-1,2,4-triazin-3(2H)-one (4c) in 20 mL of xylene; adding 1.1 mmol of 1,3-propane sultone into the solute under stirring; slowly heating to 100˜110° C. for thermal insulation reaction; monitoring with TLC until complete transformation of the substrate; and then cooling the reaction system to room temperature; evaporating part of solvent from the reaction system under reduced pressure; precipitating solid from the cooling system, filtering under diminished pressure; washing the solid with a small amount of cold ethanol, and vacuum drying to obtain the target compound IV-5. The yield of compound IV-5 is 81%. MS(ESI) m/z 396.35 (M+H)+, calcd. for C₁₆H₂₁N₅O₅S m/z=395.13.

### Example 15

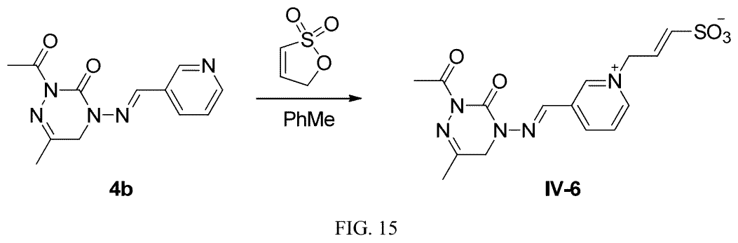

As shown in FIG. 15, example 15 provided a sulfonyl functional compound (IV-6) and it's preparing method.

The method included: dissolving 1 mmol of substrate, 2-acetyl-6-methyl-4-((pyridin-3-ylmethylene)amino)-4,5-dihydro-1,2,4-triazin-3(2H)-one (4b) in 20 mL of toluene; adding 1.1 mmol of prop-1-ene-1,3-sultone into the solute under stirring; slowly heating to 100˜110° C. for thermal insulation reaction; monitoring with TLC until complete transformation of the substrate; and then cooling the reaction system to room temperature; evaporating large part of solvent from the reaction system under reduced pressure; cooling and filtering under diminished pressure; washing the solid from the filter residue with a small amount of cold ethanol, and vacuum drying to obtain the target compound IV-6.

The yield of compound IV-6 is 92%. MS(ESI) m/z 380.36 (M+H)+, calcd. for C₁₅H₁₇N₅O₅S m/z=379.10.

### Example 16

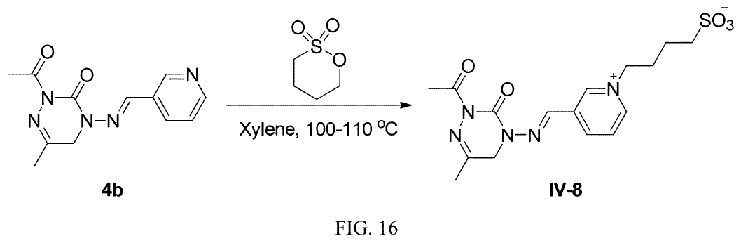

As shown in FIG. 16, example 16 provided a sulfonyl functional compound (IV-8) and it's preparing method.

The method included: dissolving 1 mmol of substrate, 2-acetyl-6-methyl-4-((pyridin-3-ylmethylene)amino)-4,5-dihydro-1,2,4-triazin-3(2H)-one (4b) in 20 mL of xylene; adding 1.1 mmol of 1,4-butane sultone into the solute under stirring; slowly heating to 100˜110° C. for thermal insulation reaction; monitoring with TLC until complete transformation of the substrate; and then cooling the reaction system to room temperature; evaporating large part of solvent from the reaction system under reduced pressure; cooling and filtering under diminished pressure; washing the solid from the filter residue with a small amount of cold acetone, and vacuum drying to obtain the target compound IV-8. The yield of compound IV-8 is 86%. MS(ESI) m/z 396.29 (M+H)+, calcd. for C₁₆H₂₁N₅O₅S m/z=395.13.

### Test for Insecticidal Activity

The insecticidal activity of sulfonyl functional compounds in the above embodiments were tested for agricultural biological functions, and the targets of insecticidal activity were Heliothis armigera, Plutella xylostella and Aphiscraccivora.

Using Heliothis armigera and Plutella xylostella as target, the insecticidal activities of sulfonyl functional compounds were tested by conventional artificial feed surface coating method. 300 microliters of artificial feed and 20 microliters of test solution were added to each well of the 24-well cell culture plate. The test solution was used in different concentrations and repeated twice (Rep1 and Rep2). The concentrations of the test solution were 500 mg/L, 250 mg/L and 125 mg/L, respectively.

The insecticidal activity of sulfonyl functional compounds against Aphiscraccivora was tested by the method of dipping. Mother liquors were prepared by the sulfonyl functional compounds provided in the above embodiments dissolved in ethanol, respectively. 500 mg/L, 250 mg/L or 125 mg/L test solution could be prepared by diluting the different mother liquors with a liquor that contained 0.1 mL/L of emulgator (Tween-80). Every test solution was tested twice (Rep1 and Rep2). A blank control was set up, and the activity results were compared with the blank control. The mortality rate of 0˜30%, 30˜50%, 50-70%, 70˜90%, or 90˜100% could be orderly set as 0, 3, 5, 7, or 9.

Some test results are shown in Table 1 below. The number of compounds in Table 1 correspond to those in the example 1˜16. “a” refers to the target, and “b” refers to the concentration of test solution (unit: mg/L).

TABLE 1
Insecticidal activity of sulfonyl functional compounds

| | Heliothis | | | | Plutella | | | | | | |
|---|---|---|---|---|---|---|---|---|---|---|---|
| Compd. | armigera ^{a} | | | Compd. | xylostella ^{a} | | | Compd. | Aphiscraccivoraª | | |
| No. | 500^{b} | 250^{b} | 125^{b} | No. | 500^{b} | 250^{b} | 125^{b} | No. | 500^{b} | 250^{b} | 125^{b} |
| I-1 | 3 | 0 | 0 | I-1 | 5 | 0 | 0 | I-1 | 9 | 7 | 5 |
| I-2 | 5 | 3 | 0 | I-2 | 5 | 3 | 0 | I-2 | 9 | 9 | 7 |
| I-3 | 3 | 3 | 0 | I-3 | 5 | 5 | 3 | I-3 | 9 | 7 | 7 |
| I-4 | 3 | 3 | 0 | I-4 | 5 | 3 | 0 | I-4 | 7 | 5 | 3 |
| I-5 | 3 | 0 | 0 | I-5 | 5 | 5 | 3 | I-5 | 7 | 3 | 3 |
| I-6 | 3 | 0 | 0 | I-6 | 5 | 3 | 0 | I-6 | 7 | 7 | 5 |
| II-1 | 0 | 0 | 0 | II-1 | 3 | 0 | 0 | II-1 | 3 | 0 | 0 |
| II-4 | 3 | 0 | 0 | II-4 | 5 | 3 | 0 | II-4 | 3 | 3 | 0 |
| III-1 | 0 | 0 | 0 | III-1 | 0 | 0 | 0 | III-1 | 0 | 0 | 0 |
| III-2 | 0 | 0 | 0 | III-2 | 0 | 0 | 0 | III-2 | 0 | 0 | 0 |
| III-3 | 0 | 0 | 0 | III-3 | 0 | 0 | 0 | III-3 | 0 | 0 | 0 |
| IV-1 | 3 | 3 | 0 | IV-1 | 5 | 3 | 3 | IV-1 | 9 | 9 | 9 |
| IV-2 | 0 | 0 | 0 | IV-2 | 5 | 3 | 0 | IV-2 | 9 | 9 | 7 |
| IV-3 | 3 | 0 | 0 | IV-3 | 3 | 3 | 0 | IV-3 | 9 | 9 | 9 |
| IV-4 | 5 | 3 | 0 | IV-4 | 5 | 5 | 3 | IV-4 | 9 | 9 | 9 |
| IV-5 | 0 | 0 | 0 | IV-5 | 3 | 0 | 0 | IV-5 | 9 | 7 | 7 |
| IV-6 | 3 | 3 | 0 | IV-6 | 5 | 3 | 0 | IV-6 | 9 | 9 | 5 |
| IV-7 | 0 | 0 | 0 | IV-7 | 3 | 0 | 0 | IV-7 | 9 | 7 | 5 |
| IV-8 | 0 | 0 | 0 | IV-8 | 3 | 3 | 0 | IV-8 | 9 | 9 | 9 |
| IV-9 | 0 | 0 | 0 | IV-9 | 3 | 0 | 0 | IV-9 | 9 | 7 | 7 |
| CK | 0 | 0 | 0 | CK | 0 | 0 | 0 | CK | 0 | 0 | 0 |

As shown in Table 1, some of the tested sulfonyl functional compounds have different degrees of insecticidal activity against Heliothis armigera, Plutella xylostella and Aphiscraccivora at the tested concentrations. In particular, some sulfonyl functional compounds show significant killing activity against Aphiscraccivora at the three test concentrations, and the death rate of Aphiscraccivora could reach more than 90%. The above results indicate that these sulfonyl functional compounds have obvious insecticidal activity. It is found that these compounds have excellent water solubility, which provides favorable basic conditions for developing the application technology of these compounds. The discovery of this new functional derivative of sulfonic acid can also be one of the means to solve the existing insecticide resistance.

### Test for Bactericidal Activity

The bactericidal activity of sulfonyl functional compounds in the above embodiment were also tested. And the targets of insecticidal activity were plant pathogenic fungi such as Verticillium dahliae.kieb., Fusarium culmorum, Fusarium oxysporum. sp. Niveum, Thanatephorus cucumeris, Septoria nodorumBerk., Botrytis cinerea, Leptosphaeria biglobosa, Alternaria kikuchiana, Corynespora leaf spot of tomato, Rhizoctonia solani, Magnaporthe oryzae, Colletotrichum gloeobosporioids, Pythium aphanidermatum.

Tests of bactericidal activity were performed by microdilution. Test solutions could be prepared by dissolving the sulfonyl functional compounds in the above embodiment in DMSO. The test solutions were shifted to a aseptic 96 well plate, diluted to 100 g/mL, 50 g/mL, 25 g/mL or 12.5 g/mL per well by double tandem dilution. And the final concentration of DMSO per well was controlled no more than 5%. Bactericidal activity was measured by inoculation medium and aseptic water containing 5% DMSO as a negative control. The fungi were cultured at 28° C. for 48-96 h. The growth status of these fungi was observed. And the results of bactericidal activity were collected and compared with blank control.

### Evaluation Criteria:

The antibacterial rate of 0˜30% could be set as 3 if the growth of the target is normal and consistent with that of the blank control. The antibacterial rate of 30˜70% could be set as 5 if the target has partial growth. The antibacterial rate of 70˜90% could be set as 7 if the target has small amounts of growth. The antibacterial rate of 90˜100% could be set as 9 if the target has totally no growth.

The results of bactericidal activity of compounds III-1, III-2 and III-3 are shown in Table 2 below. “a” refers to the target, and “b” refers to the concentration of test solution (unit: mg/L).

TABLE 2
Fungicidal activity of compounds III-1, III-2 and III-3

| | Activity | | | | | | | | | | | |
|---|---|---|---|---|---|---|---|---|---|---|---|---|
| | III-1ª | | | | III-2ª | | | | III-3ª | | | |
| Target | 100^{b} | 50^{b} | 25^{b} | 12.5^{b} | 100 | 50 | 25 | 12.5 | 100 | 50 | 25 | 12.5 |
| Verticillium dahliae. kieb. | 9 | 9 | 9 | 9 | 9 | 9 | 9 | 9 | 9 | 9 | 9 | 9 |
| Fusarium culmorum | 9 | 9 | 9 | 9 | 9 | 9 | 9 | 7 | 9 | 9 | 9 | 7 |
| Fusarium oxysporum f. sp. | 9 | 9 | 9 | 9 | 9 | 9 | 9 | 9 | 9 | 9 | 9 | 9 |
| Niveum | | | | | | | | | | | | |
| Thanatephorus cucumeris | 9 | 9 | 9 | 9 | 9 | 9 | 9 | 9 | 9 | 9 | 9 | 9 |
| Septoria nodorum Berk. | 9 | 9 | 9 | 9 | 9 | 9 | 9 | 9 | 9 | 9 | 9 | 9 |
| Botrytis cinerea | 3 | 3 | 3 | 3 | 3 | 3 | 3 | 3 | 3 | 3 | 3 | 3 |
| Leptosphaeria biglobosa | 9 | 9 | 9 | 9 | 9 | 9 | 9 | 9 | 9 | 9 | 9 | 9 |
| Alternaria kikuchiana | 3 | 3 | 3 | 3 | 3 | 3 | 3 | 3 | 3 | 3 | 3 | 3 |
| Corynespora leaf spot of | 7 | 7 | 3 | 3 | 7 | 7 | 3 | 3 | 7 | 7 | 3 | 3 |
| tomato | | | | | | | | | | | | |
| Rhizoctonia solani | 3 | 3 | 3 | 3 | 3 | 3 | 3 | 3 | 3 | 3 | 3 | 3 |
| Colletotrichum | 9 | 9 | 9 | 9 | 9 | 9 | 9 | 9 | 9 | 9 | 9 | 9 |
| gloeobosporioids | | | | | | | | | | | | |
| Pythium aphanidematum | 3 | 3 | 3 | 3 | 3 | 3 | 3 | 3 | 3 | 3 | 3 | 3 |
| Magnaporthe oryzae | 9 | 9 | 9 | 9 | 9 | 9 | 9 | 9 | 9 | 9 | 9 | 9 |

As shown in Table 2, compounds II-1, III-2 and III-3 have fungicidal activities against many kinds of pathogenic fungi, and the inhibition rates exceed 90%. In particular, under the low-test concentration of 12.5 g/mL, a variety of pathogenic fungi almost does not grow, and the inhibition rate could still reach more than 90%. These results indicate that the discovery of this new sulfonyl functional derivatives will provide a new solution for the integrated control of crop diseases, and also be used to solve the existing fungicide resistance.

The above is only the preferred embodiments of this disclosure and is not intended to limit this disclosure. Any modification, equivalent replacement, improvement, etc., made within the spirit and principle of this disclosure shall be included in the scope of this disclosure.

What is claimed is:

1. A compound selected from the group consisting of: 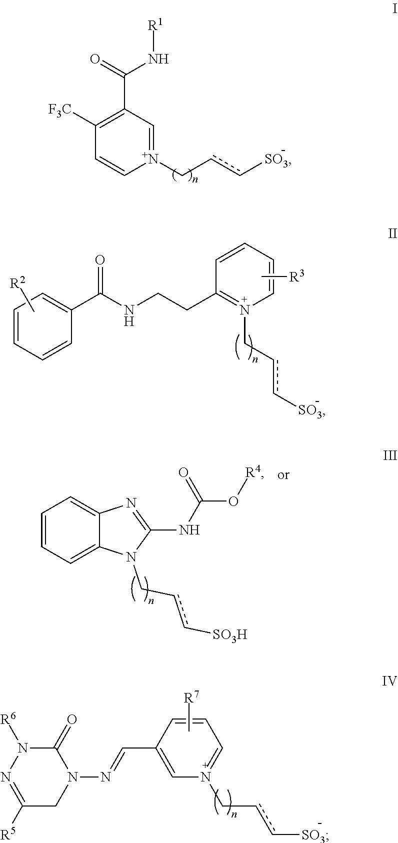
wherein, R1 is independently selected from the group consisting of CH₂CN, 4-CF₃OPhCH₂CH₂, 4-CF₃PhCH₂CH₂, 4-FPhCH₂CH₂, 4-CF₃OPhCH₂, CH₂CO₂Me, CH₂CONH₂, CH(CH₃)CO₂Me, CH(CH₃)CONH₂;
R2 is independently selected from the group consisting of H, 2-CF₃, 4-CF₃, 2-F, 2-CF₃O;
R3 is independently selected from the group consisting of H, F, Cl, Br, CF₃, CF₃O;
R4 is independently selected from the group consisting of CH₃, CH₂CH₃;
R5 is independently selected from the group consisting of CH₃, CH₂CH₃, Ph, 4-ClPh, 4-FPh, 4-CF₃Ph;
R6 is independently selected from the group consisting of H, COCH₃, COCH₂CH₃, COCF₃, CO₂CH₃, CO₂CH₂CH₃, CO₂tBu;
R7 is independently selected from the group consisting of H, F, Cl, Br, CF₃, CH₃, CF₃O, CN;
n is 1 or 2.

2. The compound according to claim 1, wherein, the compound shown in formula (I) is selected from the group consisting of:

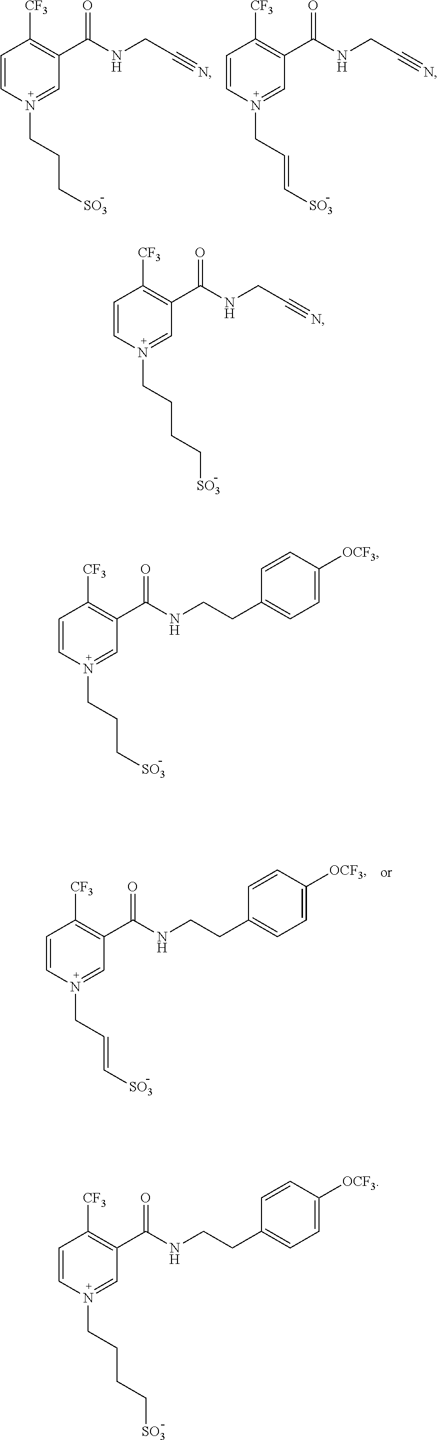

3. The compound according to claim 1, wherein, the compound shown in formula (II) is selected from the group consisting of:

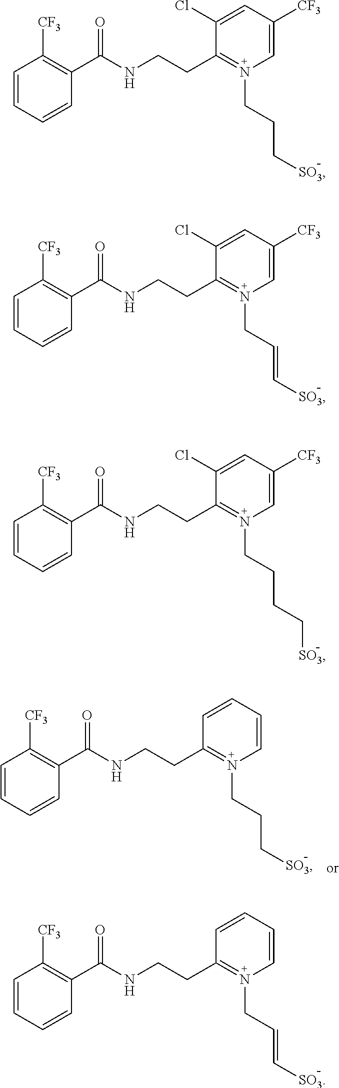

4. The compound according to claim 1, wherein, the compound shown in formula (III) is selected from the group consisting of:

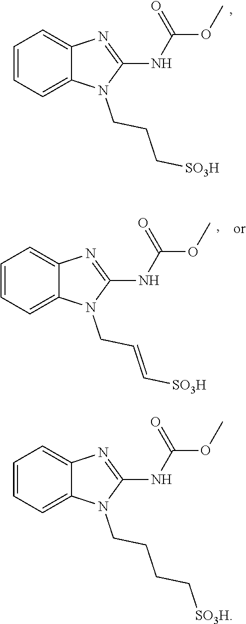

5. The compound according to claim 1, wherein, the compound shown in formula (IV) is selected from the group consisting of:

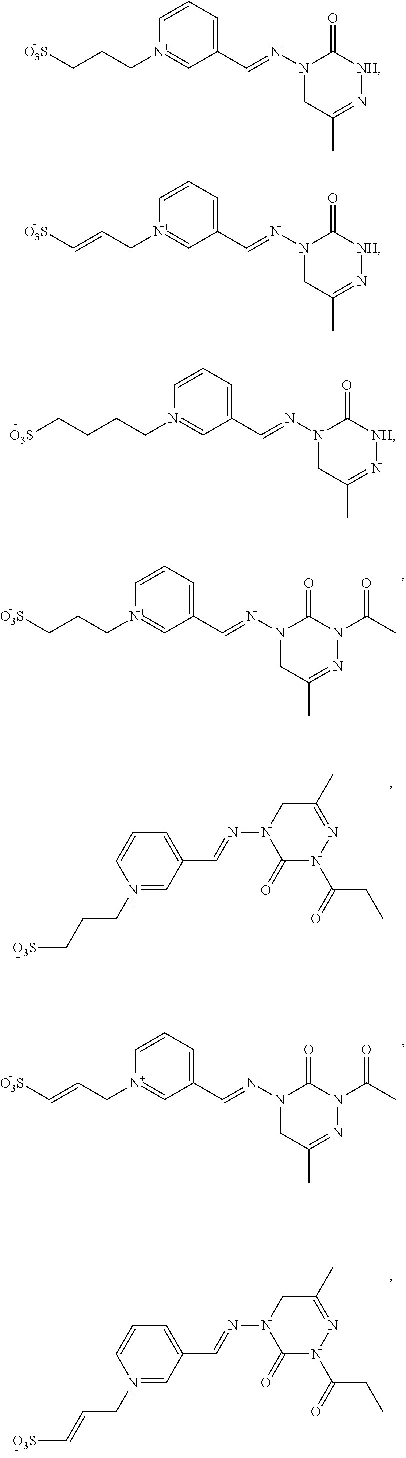

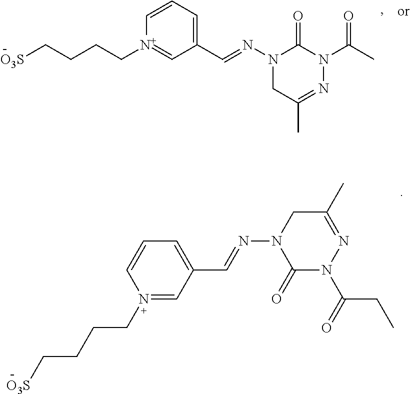

6. A composition comprising solvates or soluble salts formed by pharmaceutically acceptable solvents and a compound selected from the group consisting of: 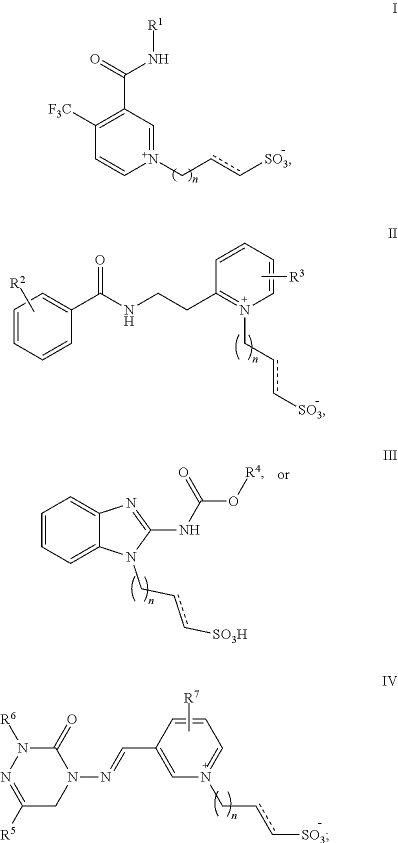
wherein, R1 is independently selected from the group consisting of CH₂CN, 4-CF₃OPhCH₂CH₂, 4-CF₃PhCH₂CH₂, 4-FPhCH₂CH₂, 4-CF₃OPhCH₂, CH₂CO₂Me, CH₂CONH₂, CH(CH₃)CO₂Me, CH(CH₃)CONH₂;
R2 is independently selected from the group consisting of H, 2-CF₃, 4-CF₃, 2-F, 2-CF₃O;
R3 is independently selected from the group consisting of H, F, Cl, Br, CF₃, CF₃O;
R4 is independently selected from the group consisting of CH₃, CH₂CH₃;
R5 is independently selected from the group consisting of CH₃, CH₂CH₃, Ph, 4-ClPh, 4-FPh, 4-CF₃Ph;
R6 is independently selected from the group consisting of H, COCH₃, COCH₂CH₃, COCF₃, CO₂CH₃, CO₂CH₂CH₃, CO₂tBu;
R7 is independently selected from the group consisting of H, F, Cl, Br, CF₃, CH₃, CF₃O, CN;
n is 1 or 2.

7. The composition according to claim 6, wherein, the compound shown in formula (I) is selected from the group consisting of:

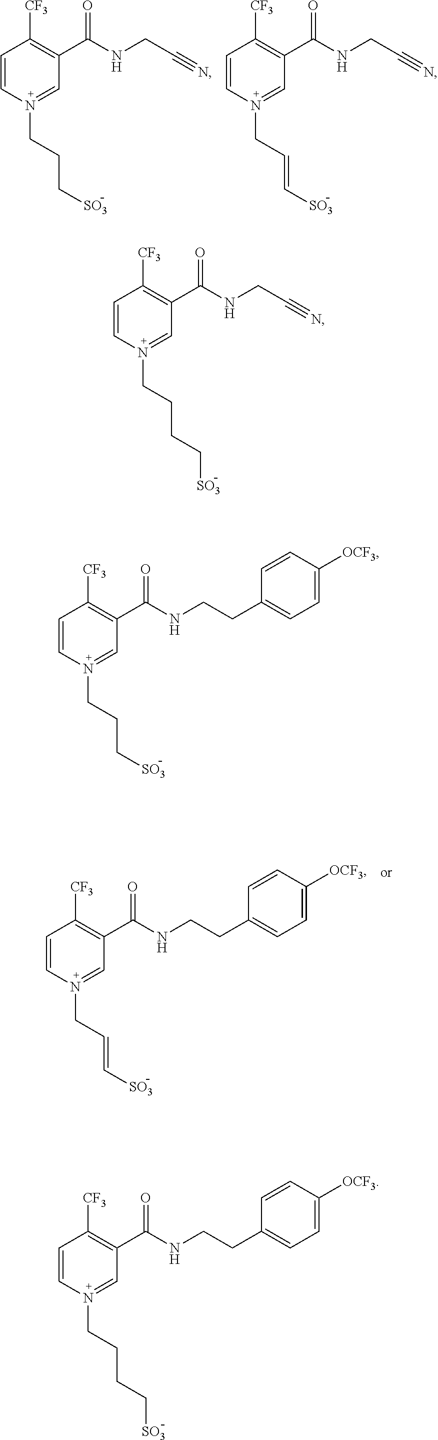

8. The composition according to claim 6, wherein, the compound shown in formula (II) is selected from the group consisting of:

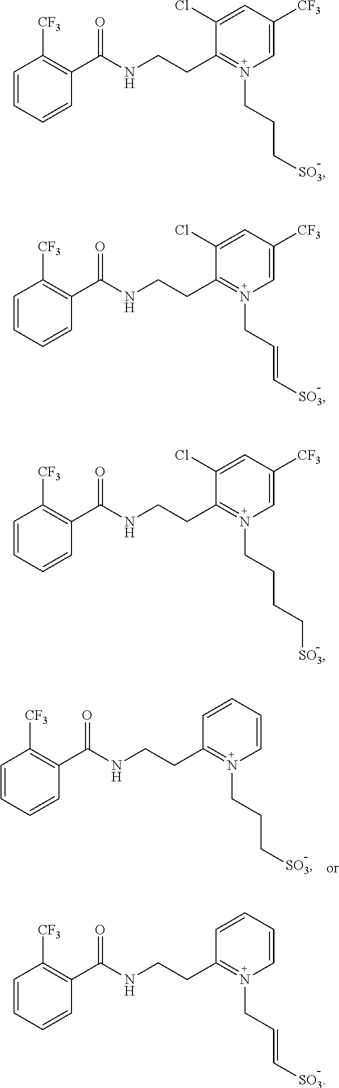

9. The composition according to claim 6, wherein, the compound shown in formula (III) is selected from the group consisting of:

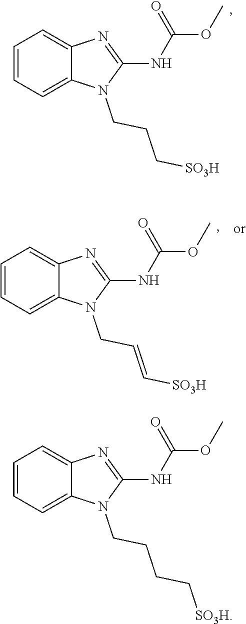

10. The composition according to claim 6, wherein, the compound shown in formula (IV) is selected from the group consisting of:

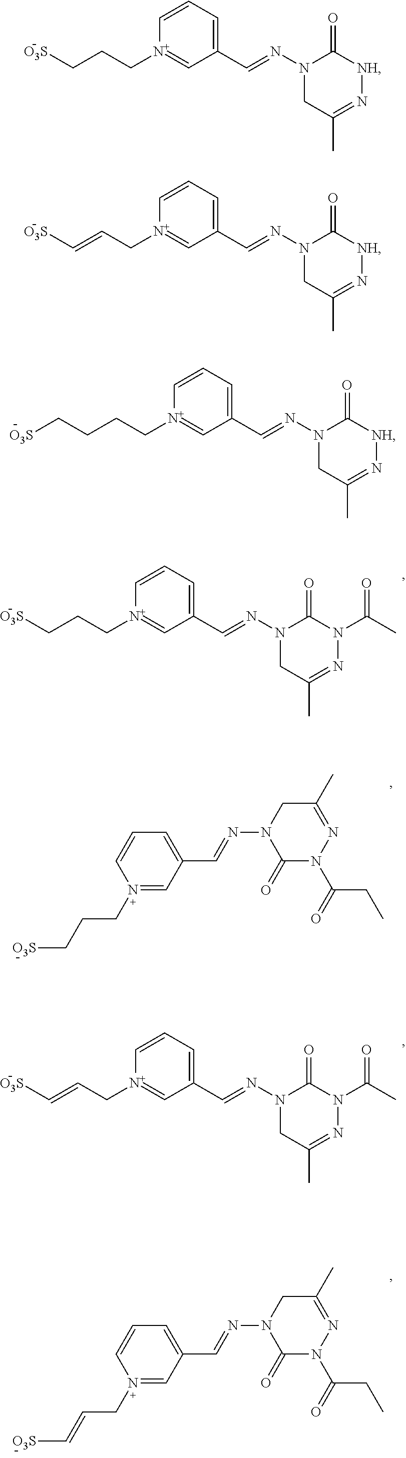

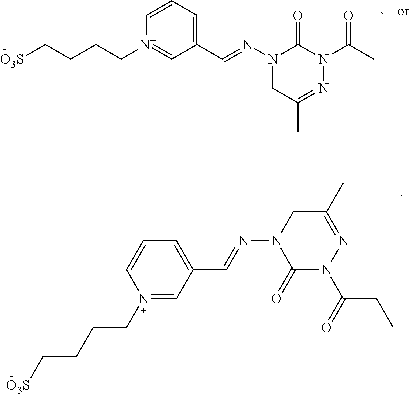

11. A pesticide comprising:
a diluter; and
a compound selected from the group consisting of 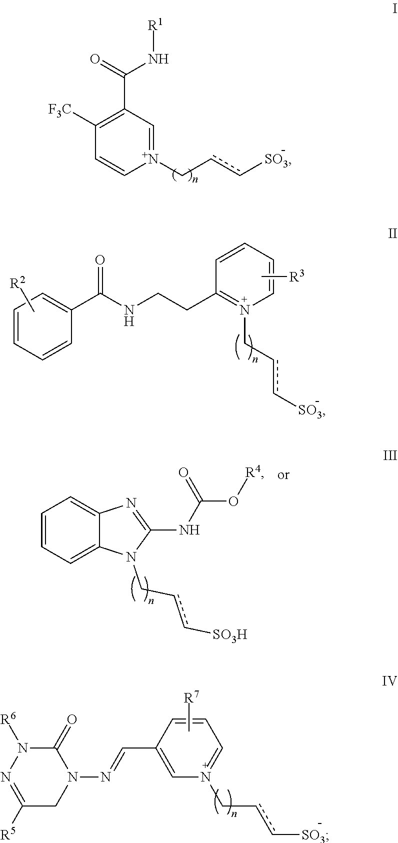
wherein, R1 is independently selected from the group consisting of CH₂CN, 4-CF₃OPhCH₂CH₂, 4-CF₃PhCH₂CH₂, 4-FPhCH₂CH₂, 4-CF₃OPhCH₂, CH₂CO₂Me, CH₂CONH₂, CH(CH₃)CO₂Me, CH(CH₃)CONH₂;
R2 is independently selected from the group consisting of H, 2-CF₃, 4-CF₃, 2-F, 2-CF₃O;
R3 is independently selected from the group consisting of H, F, Cl, Br, CF₃, CF₃O;
R4 is independently selected from the group consisting of CH₃, CH₂CH₃;
R5 is independently selected from the group consisting of CH₃, CH₂CH₃, Ph, 4-ClPh, 4-FPh, 4-CF₃Ph;
R6 is independently selected from the group consisting of H, COCH₃, COCH₂CH₃, COCF₃, CO₂CH₃, CO₂CH₂CH₃, CO₂tBu;
R7 is independently selected from the group consisting of H, F, Cl, Br, CF₃, CH₃, CF₃O, CN;
n is 1 or 2.

12. The pesticide according to claim 11, wherein, the compound shown in formula (I) is selected from the group consisting of:

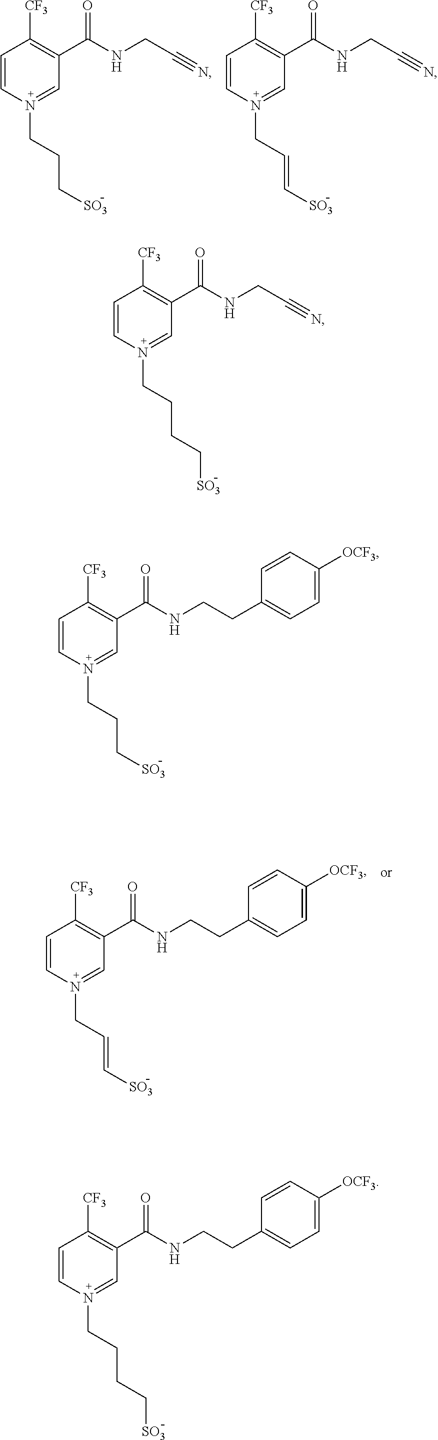

13. The pesticide according to claim 11, wherein, the compound shown in formula (II) is selected from the group consisting of:

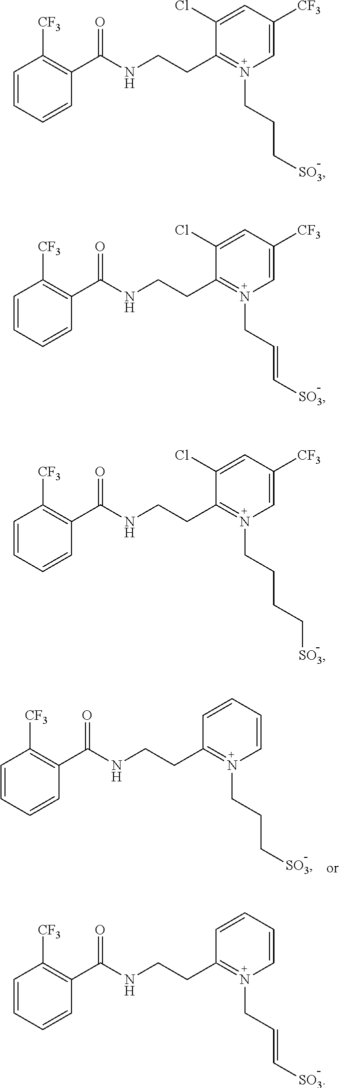

14. The pesticide according to claim 11, wherein, the compound shown in formula (III) is selected from the group consisting of:

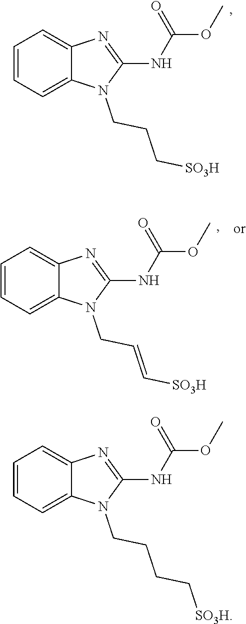

15. The pesticide according to claim 11, wherein, the compound shown in formula (IV) is selected from the group consisting of:

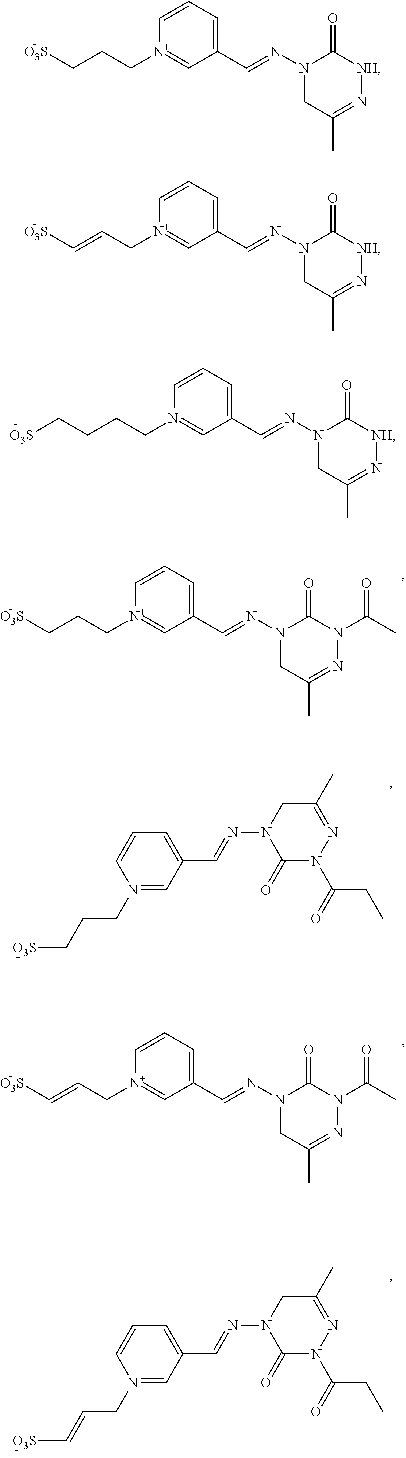

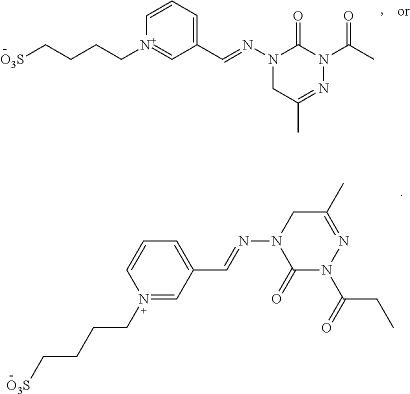

16. The pesticide according to claim 11, wherein, the diluter is selected from the group consisting of kaolin, clay, bentonite, clunch, diatomite, montmorillonite, active floridin, dolomite, quartz, calcium carbonate, talc, or magnesite.

17. The pesticide according to claim 11, wherein, the diluter is selected from the group consisting of polyvinyl alcohol, carboxymethyl cellulose, gum Arabic, trace nutrients, natural or synthetic polymers in powder, granule or emulsion form.

18. The pesticide according to claim 11, wherein, the pesticide is made into water emulsion, suspension agent, wettable powder, or water dispersion granule.
